(19) United States
(12) Patent Application Publication
(10) Pub. No.: US 2007/0238721 A1
(43) Pub. Date: Oct. 11, 2007

(54) Highly Selective Novel Amidation Method

(75) Inventors: Atsushi Inagaki, Osaka-shi (JP); Misayo Sera, Osaka-shi (JP)

Correspondence Address: EDWARDS ANGELL PALMER & DODGE LLP, P.O. BOX 55874, BOSTON, MA, 02205, US

(73) Assignee: Takeda Pharmaceutical Company Limited, Osaka (JP)

(21) Appl. No.: 11/628,906

(22) PCT Filed: Jun. 10, 2005
(86) PCT No.: PCT/JP05/11091

§ 371 Date: Dec. 8, 2006

(30) Foreign Application Priority Data
Jun. 11, 2004 (JP) 2004-174417

(51) Int. Cl. A61K 31/553; C07D 498/00

(52) U.S. Cl. 514/211.05; 540/490

(57) ABSTRACT

The present invention provides an industrial production method with a short process having a high yield of an aliphatic cyclic carboxamide having carboxyl group, which comprises reacting functional group-selectively using an inexpensive condensing agent without protecting the carboxyl group by esterification, that is, reacting carboxylic acid anhydride obtained by reacting carboxylic acid and tertiary carboxylic acid halide with aliphatic cyclic secondary amine having carboxyl group.

### TECHNICAL FIELD

The present invention relates to a novel process for producing an aliphatic cyclic carboxamide having carboxyl group.

### BACKGROUND ART

Japanese Patent No. 3479796 discloses a benzoxazepin compound which has a side-chain of aliphatic cyclic carboxamide having carboxyl group, and which is useful for preventing or treating hyperlipidemia, and in the process for producing such benzoxazepin compound, there is employed a method wherein the aliphatic cyclic secondary amine having carboxyl group is introduced by reacting an amine compound whose carboxyl group is protected by esterification under the presence of a known condensing agent (DEPC: diethyl cyanomethyl phosphonate).

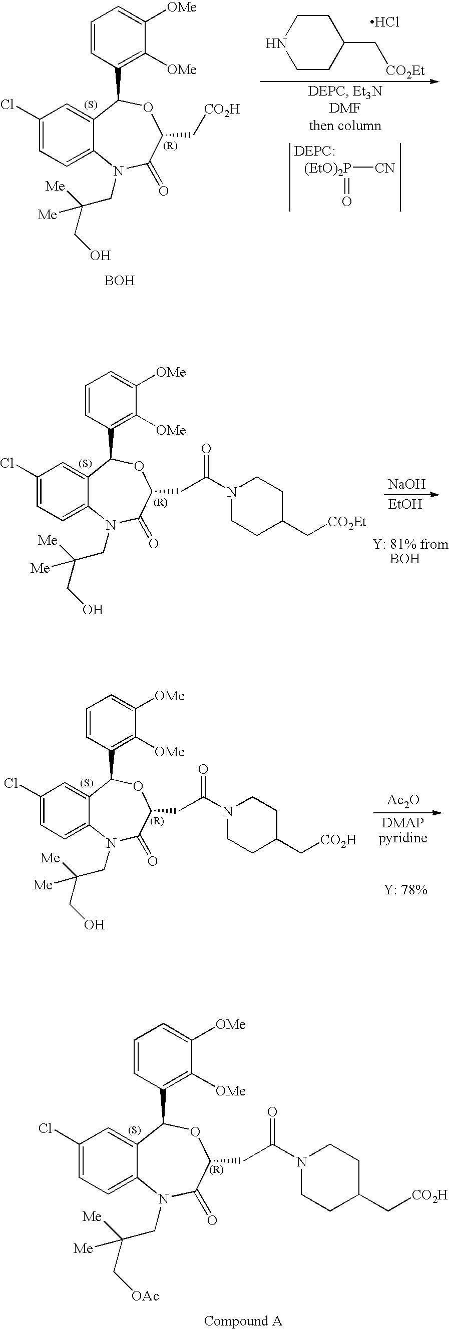

However, in this method, the condensing agent (DEPC) is expensive, and troublesome operations such as silica gel chromatography are required, and further hydrolysis of the esterified carboxyl group is needed to be carried out. Thus the method has a problem that the yield decreases by about 15% to 20%. On the other hand, a process for producing an anilide derivative having carboxyl group is described in JP 2002-80468A, and an esterified primary amine compound is used as in Japanese Patent No. 3479796, therefore, a hydrolysis operation is essential. Furthermore, in each method described in Tetrahedron, 46, 1711 (1990), Tetrahedron Lett., 30, 6841 (1989), Tetrahedron, 41, 5133 (1985), Org. Lett., 17, 3139 (2003) and Bioorg. Med. Chem. Lett., 12, 1719 (2002), the secondary amine has no carboxyl group in the molecule at all or even if it has a carboxyl group, it is protected by esterification. Thus the chemical structure of the compound of these documents is different from the aliphatic cyclic secondary amine having carboxyl group.

### DISCLOSURE OF INVENTION

### Technical Problems to be Solved by the Invention

An object of the present invention is to provide an industrial production method with a short process having a high yield of an aliphatic cyclic carboxamide having carboxyl group, which comprises chemoselective reaction using an inexpensive condensing agent without protecting the carboxyl group by esterification.

### SUMMARY OF THE INVENTION

In view of the above described problem, the present inventors have conducted intensive studies, and as a result, found out that an aliphatic cyclic carboxamide having carboxyl group of high quality can be obtained chemoselectively with high yield by reacting an aliphatic cyclic secondary amine having carboxyl group with a mixed acid anhydride formed by the reaction of a carboxylic acid (for example, a compound represented by the general formula: 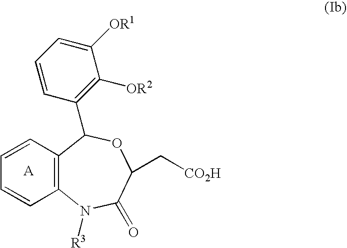
wherein R¹ and R² each independently denotes a lower alkyl group, R³ denotes a lower alkyl group which may be substituted with hydroxyl group or an alkanoyloxy group, and ring A denotes a benzene ring which may be substituted with a halogen atom, or a salt thereof) and a tertiary carboxylic acid halide, and came to the completion of the present invention.

That is, the present invention provides:

(1) A process for producing an aliphatic cyclic carboxamide having carboxyl group, which comprises reacting tertiary carboxylic acid anhydride and aliphatic cyclic secondary amine having carboxyl group,

(2) A process for producing an aliphatic cyclic carboxamide having carboxyl group, which comprises reacting carboxylic acid anhydride obtained by reacting carboxylic acid and tertiary carboxylic acid halide with aliphatic cyclic secondary amine having carboxyl group,

(3) The process according to the above-mentioned (2),

wherein the tertiary carboxylic acid halide is pivaloyl chloride,

(4) The process according to the above-mentioned (2), wherein the carboxylic acid is a compound represented by the formula: 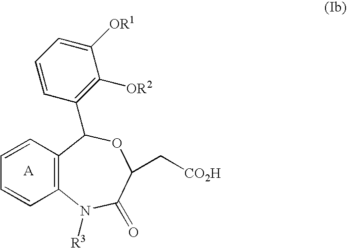
wherein R¹ and R² each independently denote a lower alkyl group, R³ denotes a lower alkyl group which may be substituted with hydroxyl group or an alkanoyloxy group, and ring A denotes a benzene ring which may be substituted with a halogen atom, or a salt thereof,

(5) The process according to the above-mentioned (2), wherein the carboxylic acid is (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid or a salt thereof,

(6) The process according to the above-mentioned (1),

wherein the aliphatic cyclic secondary amine having carboxyl group is a compound represented by the formula: 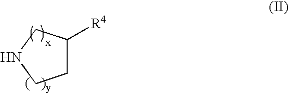
wherein x denotes an integer of 1, 2 or 3; y denotes an integer of 0, 1, or 2; and R⁴ denotes a group represented by the formula —(CH₂)_{z}—CO₂H [wherein z denotes an integer of 0, 1, 2, or 3], or a salt thereof,

(7) The process according to the above-mentioned (1), wherein the aliphatic cyclic secondary amine having carboxyl group is piperidine-4-acetic acid or a salt thereof,

(8) A process for producing 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid or a salt thereof, which comprises reacting (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic pivalic anhydride or a salt thereof with piperidine-4-acetic acid or a salt thereof,

(9) A process for producing 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid or a salt thereof, which comprises reacting (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic pivalic anhydride or a salt thereof with piperidine-4-acetic acid or a salt thereof, followed by subjecting the resulting compounds to recrystallization,

(10) A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to the above-mentioned (9), wherein the content of dipiperidyl compound is less than 0.5% of total weight of the composition,

(11) A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to the above-mentioned (9), wherein the content of dimer is less than 0.5% of total weight of the composition,

(12) A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to the above-mentioned (9), wherein the content of dimer is less than 0.3% of total weight of the composition,

(13) A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to the above-mentioned (9), wherein any impurities exceeding 0.2% of total weight of the composition other than dipiperidyl compound or dimer are not contained,

(14) A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to the above-mentioned (9), wherein the content of total impurity is less than 1.0% of total weight of the composition,

(15) A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of dipiperidyl compound is less than 0.5% of total weight of the composition, to a human in need thereof,

(16) A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of dimer is less than 0.5% of total weight of the composition, to a human in need thereof,

(17) A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of dimer is less than 0.3% of total weight of the composition, to a human in need thereof,

(18) A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein any impurities exceeding 0.2% of total weight of the composition other than dipiperidyl compound or dimer are not contained, to a human in need thereof, and

(19) A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of total impurity is less than 1.0% of total weight of the composition, to a human in need thereof.

### BEST MODE FOR CARRYING OUT THE INVENTION

An explanation of the above-mentioned general formulas and definitions included in the scope of the present invention and preferred examples thereof will be given below.

The above-mentioned tertiary carboxylic acid halide used in the present invention is not particularly limited structurally, but includes a halide of carboxylic acid wherein α carbon of carboxyl group is tertiary alkyl group. For example, tertiary carboxylic acid chlorides such as tertiary C₁₋₆ alkylcarbonyl halide and the like such as pivaloyl chloride, 2,2-dimethylbutyl chloride, 2,2-dimethylvaleroyl chloride, etc are exemplified. Among them, pivaloyl chloride is preferred.

The above-mentioned “aliphatic cyclic secondary amine having carboxyl group” used in the present invention is not particularly limited structurally, but includes a saturated or unsaturated monocyclic or polycyclic amines having carboxyl group, for example, a compound represented by the above-mentioned formula (II) or a salt thereof. Specifically, examples thereof include isonipecotic acid, nipecotic acid, pipecolinic acid, 4-piperidineacetic acid, 3-piperidineacetic acid, 2-piperidineacetic acid, 4-piperidinepropionic acid, 3-piperidinepropionic acid, 2-piperidinepropionic acid, 4-piperidinebutanoic acid, 3-piperidinebutanoic acid, 2-piperidinebutanoic acid, 3-pyrrolidinecarboxylic acid, 2-pyrrolidinecarboxylic acid (proline), 3-pyrrolidineacetic acid, 2-pyrrolidineacetic acid, 3-pyrrolidinepropionic acid, 2-pyrrolidinepropionic acid, 3-pyrrolidinebutanoic acid, 2-pyrrolidinebutanoic acid, 4-azepanecarboxylic acid, 3-azepanecarboxylic acid, 2-azepanecarboxylic acid, 4-azepaneacetic acid, 3-azepaneacetic acid, 2-azepaneacetic acid, 4-azepanepropionic acid, 3-azepanepropionic acid, 2-azepanepropionic acid, 4-azepanebutanoic acid, 3-azepanebutanoic acid, 2-azepanebutanoic acid or a salt thereof, and the like.

The above-mentioned “carboxylic acid” used in the present invention is not particularly limited structurally, but includes widely a compound having carboxyl group in the molecule. For example, a compound represented by the above-mentioned formula (Ib) or a salt thereof is exemplified.

In formula (Ib) above, the lower alkyl group represented by R¹ and R² includes a C₁₋₆ alkyl group such as methyl, ethyl, n-propyl, isopropyl, n-butyl, t-butyl, pentyl, hexyl, etc. In particular, a C₁₋₃ alkyl group is preferred. As R¹ and R², methyl group is particularly preferred from an aspect of pharmacological activity.

In formula (Ib) above, the “lower alkyl group” in the “lower alkyl group which may be substituted with hydroxyl group or an alkanoyloxy group” represented by R³ includes, for example, n-propyl, isopropyl, 1,1-dimethylethyl, n-butyl, isobutyl, n-pentyl, 2,2-dimethylpropyl, isopentyl, n-hexyl, isohexyl, and the like. Among them, isopropyl, 1,1-dimethylethyl, n-butyl, isobutyl, 2,2-dimethylpropyl and isohexyl is preferred, and 2,2-dimethylpropyl is preferred in particular.

Examples of the “alkanoyloxy group” in the “lower alkyl group which may be substituted with hydroxyl group or an alkanoyloxy group” represented by R³ include a C₁₋₂₀ alkanoyloxy group such as formyloxy, acetoxy, propionyloxy, butyryloxy, t-butoxycarbonyloxy, isobutyryloxy, valeryloxy, pivaloyloxy, lauryloxy, palmitoyloxy, stearoyloxy, etc. (preferably, C₁₋₇ alkanoyloxy group). Among them, acetoxy, propionyloxy, t-buthoxycarbonyloxy, and palmitoyloxy is preferred, and in particular, acetoxy is preferred. One to three of alkanoyloxy group or hydroxyl group may be substituted at substitutable positions. Preferred examples of the lower alkyl group which may be substituted with hydroxyl group or an alkanoyloxy group represented by R³ include 2,2-dimethylpropyl, 3-hydroxy-2,2-dimethylpropyl, 3-hydroxy-2-hydroxymethyl-2-methylpropyl, 3-acetoxy-2,2-dimethylpropyl, 3-acetoxy-2-hydroxymethyl-2-methyl-propyl and 3-acetoxy-2-acetoxymethyl-2-methylpropyl. Among them, 2,2-dimethylpropyl is particularly preferred. In addition, as R³, a lower alkyl group having an alkanoyloxy group and/or hydroxyl group is preferred.

In formula (Ib) above, the halogen atom which may be substituted in ring A includes, for example, chlorine, fluorine, bromine, and iodine atom, and in particular, the chlorine atom is preferred.

Compound (Ib) may be any one of a free compound or a salt thereof, which is included in the present invention. As such salt, in the case where compound (Ib) has an acidic group such as carboxyl group, it may form a salt with an inorganic base (for example, alkali metals such as sodium, potassium, etc., alkaline earth metals such as calcium, magnesium, etc., a transition metals such as zinc, iron, copper, etc., and the like) or an organic base (for example, organic amines such as trimethylamine, triethylamine, pyridine, picoline, ethanolamine, diethanolamine, triethanolamine, dicyclohexylamine, N,N′-dibenzylethylenediamine, etc., basic amino acids such as arginine, lysine, ornithine, etc.).

Compound (Ib) or a salt thereof may be either of hydrate and non-hydrate. In addition, compound (Ib) or a salt thereof may be labeled with an isotopic element (e.g., ³H, ¹⁴C, ³⁵S, ¹²⁵I and the like).

The compound represented by formula (Ib) or a salt thereof has asymmetric carbons at 3-position and 5-position, therefore the compound may be a mixture of stereoisomers or a separated stereoisomer. Each of the stereoisomers can be separated from a mixture thereof with known means. The trans isomer, which is an isomer in which the substituents of 3-position and 5-position are oriented in the opposite direction to the plane of 7-membered ring, is preferred. In particular, those in which the absolute configuration of 3-position is R configuration and the absolute configuration of 5-position is S configuration are preferred. In addition, it may be a racemic compound or an optically active compound. The optically active compound can be separated from the racemic compound by a known optical resolution mean.

Examples of the above-mentioned “aliphatic cyclic carboxamide having carboxyl group” used in the present invention include widely a compound formed by a condensation of the above-mentioned “carboxylic acid” and the “aliphatic cyclic secondary amine having carboxyl group” with forming an amide bond, a salt thereof. For example, a compound represented by the following formula (I) or a salt thereof; 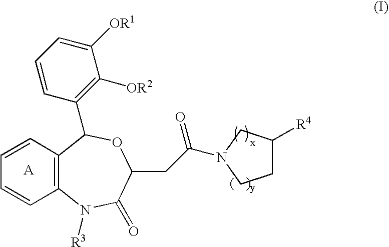
(wherein each symbol is as defined above)
a compound wherein the moiety of aliphatic cyclic secondary amine having carboxyl group is piperidyl group having carboxyl group (e.g. Argatroban, compound of development number: (+)-NSL-95301 ((+)-2-[1-[3-(4-amidinobenzamido)-2,2-dimethyl-3-phenylpropionyl]piperidin-4-yl]acetic acid), etc.); a compound wherein the moiety of aliphatic cyclic secondary amine having carboxyl group is pyrrolidinyl group having carboxyl group (e.g. Enalapril, Captopril, etc.) and the like are exemplified.

As a reactive derivative of carboxyl group for amidation, for example, an acid anhydride, mixed acid anhydride, acid chloride, imidazole derivative and the like are used generally. However, in the production of aliphatic cyclic carboxamide such as 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid (hereinafter, referred to as “compound A” in the present specification) and the like, the formation of by-product (in the case of compound A, (3R,5S)-7-chloro-5-(2,3-dimethoxyphenyl)-1,2,3,5-tetrahydro-1-(3-acetoxy-2,2-dimethylpropyl)-2-oxo-4,1-benzoxazepin-3-acetatic acid (BOA: raw material)) is unexpectedly inhibited when an activating agent having a bulky substituent such as tertiary carboxylic acid halide (for example, trimethylacetyl chloride (another name: pivaloyl chloride)) is used as an activating agent, and a high reaction progress rate (92%) can be obtained (refer to Table 1).

TABLE 1

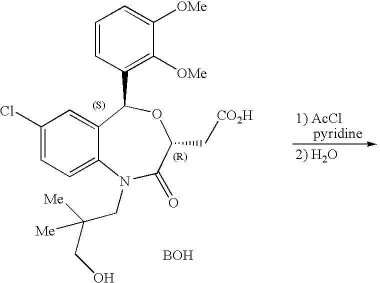

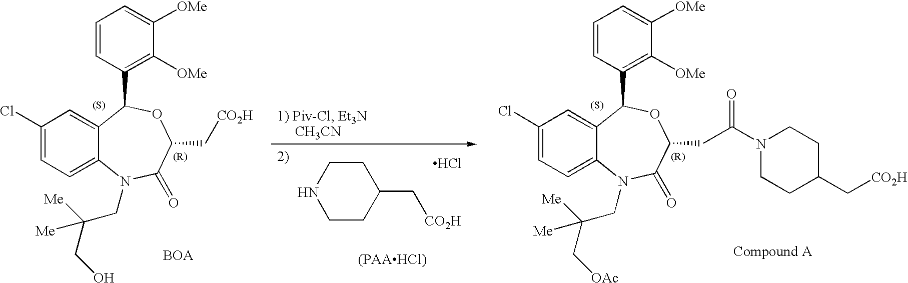

Activating agent¹⁾ of synthetic reaction of compound A

Yield (%)²⁾

| run | Activating agent | Compound A | BOA |
|---|---|---|---|
| 1 | SOCl₂ | 61 | 19 |
| 2 | CDI³⁾ | 86 | 1 |
| 3 | ClCO₂Ph | 55 | 22 |
| 4 | ClCO₂Allyl | 46 | 29 |
| 5 | ClCO₂Me | 54 | 36 |
| 6 | ClCO₂Et | 68 | 27 |
| 7 | ClCO₂iBu | 71 | 24 |
| 8 | ClCO₂iPr | 81 | 11 |
| 9 | ClCOCMe₃ | 92 | 2 |

¹⁾Reaction condition: CH₃CN, DBU, r.t., 2h
²⁾Reaction solution is measured by HPLC (area of HPLC)
³⁾N,N′-carbonyldiimidazole

The reaction between the above-mentioned compound presented by general formula (Ib) and compound represented by general formula (II) in the present invention is carried out, for example, by adding 1 to 10 fold moles, preferably, 1 to 2 fold moles of base and tertiary carboxylic acid halide to 1 mole of the compound represented by general formula (Ib), and reacting at a reaction temperature of −20° C. to 50° C., preferably, −10° C. to 1° C. for a reaction time of 0.1 to 10 hours, preferably, 0.2 to 2 hours. Examples of the base include inorganic bases such as potassium carbonate, sodium carbonate, potassium hydrogen carbonate, sodium hydrogen carbonate, potassium tert-butoxide, sodium hydroxide, potassium hydroxide, lithium hydroxide, etc., and organic bases such as triethylamine, diisopropylethylamine, 4-dimethylaminopyridine, triethylenediamine, tetramethylethylenediamine, 1,8-diazabicyclo[5.4.0]undeca-7-ene (abbreviation: DBU), etc.

Reaction is carried out in a proper solvent. As the solvent, for example, water, alcohols such as methanol, ethanol, n-propanol, isopropanol, etc., aromatic hydrocarbons such as benzene, toluene, xylene, etc., halogenated hydrocarbons such as dichloromethane, chloroform, etc., ethers such as diethyl ether, tetrahydrofuran, dioxane, etc., ketones such as acetone, methyl ethyl ketone, etc., nitriles such as acetonitrile, etc., sulfoxides such as dimethylsulfoxide, etc., acid amides such as N,N-dimethylformamide, N,N-dimethylacetamide, etc., esters such as ethyl acetate, etc., and carboxylic acids such as acetic acid, propionic acid, etc. can be used. These solvents may be used alone or, if needed, by mixing two or more at an appropriate ratio, for example, at a ratio of 1:1 to 1:10. In this reaction, a base and pivaloyl chloride may be added individually and sequentially, or added simultaneously.

The obtained tertiary carboxylic acid anhydride can be isolated and purified by known isolating and purifying methods, for example, concentration, concentration under reduced pressure, extraction with solvent, crystallization, recrystallization, transfer dissolution, chromatography and the like, however it can be reacted with the compound represented by general formula (II) without being isolated or purified. For example, 1 to 10 fold moles, preferably, 1 to 2 fold moles of the compound represented by general formula (II) (e.g., 4-piperidineacetic acid hydrochloride) and base is added to 1 mole of the compound represented by general formula (Ib), and the reaction is carried out at a reaction temperature of −20° C. to 50° C., preferably, −10° C. to 10° C. for a reaction time of 0.1 to 10 hours, preferably, 0.5 to 5 hours. As the base, inorganic bases or organic bases is used as described above. The reaction is carried out in an appropriate solvent, and said solvent includes those above-mentioned. In the reaction, the compound represented by general formula (II) or a salt thereof and the base may be added sequentially to a solvent, or alternatively a mixture in an appropriate solvent of the compound represented by general formula (II) or a salt thereof and the base prepared separately may be added to a solvent.

The aliphatic cyclic carboxamide having carboxyl group obtained in this reaction can be isolated and purified by a simple operation such as concentration, concentration under reduced pressure, crystallization, recrystallization, and the like.

When the “aliphatic cyclic carboxamide having carboxyl group” obtained by the production method of the present invention is compound A, the compound A can be isolated as crystals efficiently with a convenient operation of adding, for example, n-heptane (preferably under warming) to the organic layer after the completion of reaction, which is based on the high yield of compound A in the reaction. The conditions such as amount of n-heptane to be added, temperature at the addition and the like can be selected appropriately.

For example, 0.1 to 10.0 fold amount (v/v), preferably, 0.5 to 2.0 fold amount (v/v) of n-heptane is added to the organic layer after the completion of reaction at a temperature of 20° C. to 90° C., preferably, 40° C. to 80° C. The resulting crude crystals can be further purified highly by dissolving again in ethyl acetate and adding n-heptane thereto. When dissolving, the solubility of the crude crystals can be enhanced by adding 0.1 to 5.0 fold amount (v/w), preferably, 0.5 to 1.0 fold amount (v/w) of water or ethanol relative to the crude crystals.

Furthermore, compound A can be obtained as crystals having an extremely high purity by recrystallizing the crude crystals from a mixed solvent of alcohol (e.g., ethanol, etc.) and water. The conditions such as mixing ratio of alcohol and water, temperature for crystallization, times of recrystallization, and the like can be selected appropriately. For example, 3 to 50 times (v/w), preferably, 5 to 10 times (v/w) the amount of hydrous alcohol relative to the crude crystals is added to dissolve, and 1 to 100 times (v/w), preferably, 5 to 10 times (v/w) the amount of water is added thereto at a temperature of 20° C. to 100° C., preferably, 40° C. to 70° C. The water content of hydrous alcohol is 0 to 90%, preferably, 5 to 20%.

Compound A or a salt thereof obtained by the production method and recrystallization of the present invention is obtained as a composition containing less than 0.5% of total weight of the composition (preferably less than 0.4%, more preferably less than 0.3%, further more preferably less than 0.2%) of the compound represented by formula (III) (hereinafter, referred to as dipiperidyl compound in some cases). In addition, it is obtained as a composition containing less than 0.5% of total weight of the composition (preferably less than 0.3%, more preferably less than 0.2%, further more preferably less than 0.1%) of the compound represented by formula (IV) (hereinafter, referred to as dimmer in some cases).

Therefore, from the viewpoint of the content of compound A, a preferable composition wherein the content of compound A in the composition is 99.0% (W/W) or more (i.e. the content of total impurity is less than 1.0%) (more preferably, 99.5% or more (i.e. the content of total impurity is less than 0.5%)) can be obtained by using the production method of the present invention, and from the viewpoint of the content of impurities, a preferable composition of compound A which contains no impurities exceeding 0.2% of total weight of the composition other than dipiperidyl compound or dimer (for example, this means that when 1 or 2 or more impurities are contained, the content of each of the impurities does not exceed 0.2%.) can be obtained by using the production method of the present invention.

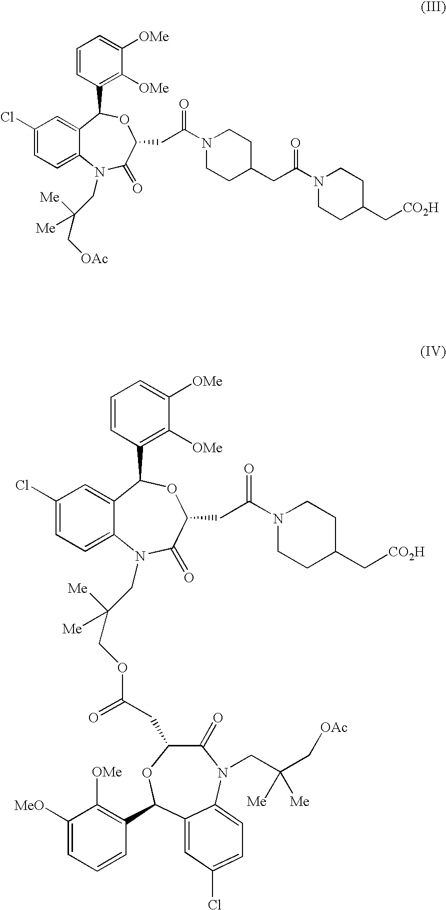

It becomes possible to produce a benzoxazepin compound and the like having higher quality by controlling the content of impurities such as dipiperidyl compound, and with the improvement of purity, improvement of the degree of crystallization, improvement of stability and the like can be expected. Furthermore, in the case where an aliphatic cyclic carboxamide having carboxyl group is used as a medicine, it is extremely important to reduce impurities from the viewpoint of quality assurance to patients. Thus compound A which is available as a medicine for clinical use can be produced efficiently by the production with the process for production and recrystallization thereafter of the present invention.

Here, compound (I) as represented by compound A is useful as squalene synthetase inhibitor, and is known to be useful for preventing and/or treating hyperlipidemia, familial hypercholesterolemia, organ failure or organ dysfunction and for protecting skeletal muscle, and the like (for example, JP 09-136880A, etc.).

The compound represented by formula (Ib) or a salt thereof can be produced by a method disclosed in, for example, EP567026A, WO95/21834 (PCT application based on JP Application No. H06-15531), EP645377A (application based on JP Application No. H06-229159), EP645378A (application based on JP Application No. H06-229160) or analogous methods thereto.

In this case, the racemic compound of compound (Ib) or a salt thereof can be obtained by a method described in, for example, WO95/21834 or an analogous method thereto. The optically active isomers of compound (Ib) or a salt thereof can be obtained by a per se known optical resolution method or an analogous method thereto, for example, by reacting the racemic compound with an optically active amino acid ester or a derivative thereof to form an amide bond, followed by subjecting to distillation, recrystallization, column chromatography and the like to separate and purify the optically active isomer, and then, severing the amide bond again.

Alternatively, for example, (3R,5S) compound of the above-mentioned compound (Ib) or a salt thereof may be prepared by obtaining an optically active isomer (S compound) of benzyl alcohol derivative by an enzymatic asymmetric hydrolysis with a process represented by the formula 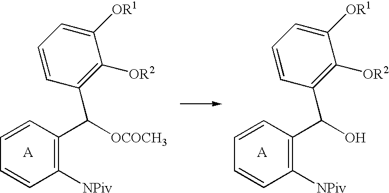
wherein Piv denotes pivaloyl group, and other symbols are as defined above, and then according to the method described in EP567026A using this optically active isomer as starting material.

In addition, (3R,5S) compound of the above-mentioned compound (Ib) or a salt thereof may be prepared by obtaining an optically active isomer (S compound) of benzyl alcohol derivative by asymmetric reduction of the process represented by the formula 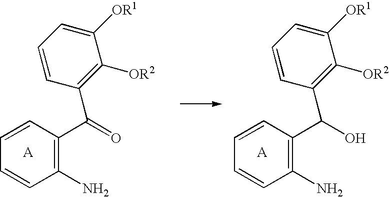
wherein symbols are as defined above, using an asymmetric reduction method described in, for example, JP 9-235255A, and then according to the method described in EP567026A using this optically active isomer as starting material.

In addition, in each reaction of the process for producing compound (Ib) or a salt thereof described above and each reaction of raw material compounds synthesis, when the raw material compound has amino group, carboxyl group or hydroxy group as a substituent, a protective group which is generally used in peptide chemistry may be introduced into these groups, and a target compound can be obtained by removing the protective group after the reaction, if needed. As the protective group for the amino group there are used, for example, formyl, C₁₋₆ alkyl carbonyl (e.g., acetyl, ethyl carbonyl, etc.), phenyl carbonyl, C₁₋₆ alkyl-oxycarbonyl (e.g., methoxycarbonyl, ethoxycarbonyl, etc.), phenyloxycarbonyl, C₇₋₁₀ aralkyl-carbonyl (e.g., benzylcarbonyl, etc.), trityl, phthaloyl, N,N-dimethylaminomethylene, or the like, each of which may have a substituent. As the substituent of these protective groups, there is used a halogen atom (e.g., fluorine, chloride, bromine, iodine, etc.), C₁₋₆ alkyl-carbonyl (e.g., methylcarbonyl, ethylcarbonyl, butylcarbonyl, etc.), nitro group and the like, and the number of substituents is about 1 to 3. As the protective group of carboxyl group, there is used, for example, C₁₋₆ alkyl (e.g., methyl, ethyl, n-propyl, i-propyl, n-butyl, tert-butyl, etc.), phenyl, trityl, silyl, or the like, each of which may have a substituent. As the substituent of these protective groups, there are used a halogen atom (e.g., fluorine, chloride, bromine, iodine etc.), formyl, C₁₋₆ alkyl-carbonyl (e.g., acetyl, ethylcarbonyl, butylcarbonyl, etc.), nitro group and the like, and the number of substituents is about 1 to 3. As the protective group of hydroxy group, there are used, for example, C₁₋₆ alkyl (e.g., methyl, ethyl, n-propyl, i-propyl, n-butyl, tert-butyl, etc.), phenyl, C₇₋₁₀ aralkyl (e.g., benzyl, etc.), formyl, C₁₋₆ alkyl-carbonyl (e.g., acetyl, ethylcarbonyl, etc.), phenyloxycarbonyl, benzoyl, C₇₋₁₀ aralkyl-carbonyl (e.g., benzylcarbonyl, etc.), pyranyl, furanyl, silyl, or the like, each of which may have a substituent. As the substituent of these protective groups, there are used a halogen atom (e.g., fluorine, chlorine, bromine, iodine, etc.), C₁₋₆ alkyl (e.g., methyl, ethyl, n-propyl, etc.), phenyl, C₇₋₁₀ aralkyl (e.g., benzyl, etc.), nitro group and the like, and the number of substituents is about 1 to 4.

In addition, as a method for removing the protective group, a known method per se or a modification thereof is used and there is employed a method to treat with, for example, acid, base, reduction, ultra-violet ray, hydrazine, phenylhydrazine, sodium N-methyldithiocarbamate, tetrabutylammonium fluoride, palladium acetate or the like. The compound (Ib) or a salt thereof obtained by the above methods can be isolated and purified with usual separation means such as re-crystallization, distillation, chromatography and the like. When the thus obtained compound (Ib) of the present invention is obtained as free compound, it can be converted to a salt according to a known method per se or a modification thereof (e.g., neutralization), and, on the contrary, when obtained as a salt, it can be converted to a free compound or another salt according to a known method per se or a modification thereof. When the obtained compound is a racemic compound, it can be separated into d-isomer and l-isomer by usual optical resolution method.

The compound (Ib) or a salt thereof has a potent squalene synthetase inhibitory activity, and is useful for preventing or treating hyperlipidemia and the like.

The present invention will be described in detail through the following Reference Examples, Examples, and Preparation Examples. However, the Present Invention is not limited to these. In addition, each abbreviation in the Examples has the following meanings:

2,3-DBA: 2,3-dimethoxybenzoic acid

DMA: 2,3-dimethoxybenzmorphoamide

CAB: p-chloroaniline

CPB: N-pivaloyl-p-chloroaniline

PABP: 5-chloro-2-pivaloylamino-2′,3′-dimethoxybenzophenone

ACBP: 2-amino-5-chloro-2′,3′-dimethoxybenzophenone (S)—BH: (S)-2-amino-5-chloro-α-(2,3-dimethoxyphenyl)benzyl alcohol

CPBA: (S)-5-chloro-2-(3-hydroxy-2,2-dimethylpropyl)amino-α-(2,3-dimethoxyphenyl)benzyl alcohol

BOE: ethyl (3R,5S)-7-chloro-1,2,3,5-tetrahydro-1-(3-hydroxy-2,2-dimethylpropyl)-5-(2,3-dimethoxyphenyl)-2-oxo-4,1-benzoxazepin-3-acetate

BOH: (3R,5S)-7-chloro-1,2,3,5-tetrahydro-1-(3-hydroxy-2,2-dimethylpropyl)-5-(2,3-dimethoxyphenyl)-2-oxo-4,1-benzoxazepin-3-acetic acid

BOA: (3R,5S)-7-chloro-5-(2,3-dimethoxyphenyl)-1,2,3,5-tetrahydro-1-(3-acetoxy-2,2-dimethylpropyl)-2-oxo-4,1-benzoxazepin-3-acetic acid

Compound A: 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid

### REFERENCE EXAMPLE 1

### 2,3-dimethoxybenzmorphoamide

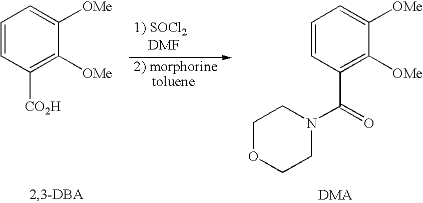

2,3-DBA (145 kg, 796 mol) was added to a mixed solution of toluene (1450 L) and N,N-dimethylformamide (0.58 kg), and thionyl chloride (113 kg, 1.2 eq) was added thereto at around 57° C. The solution was stirred for 2 hours at the same temperature. After the reaction solution was concentrated under reduced pressure up to about 500 L, toluene (1073 L) was added, and morpholine (152 kg, 2.2 eq) was added dropwise at about 10° C., and then, the solution was stirred at about 23° C. for 2 hours. City water (145 L) was added thereto, and separated the layers, and then, the aqueous later was extracted again with toluene (725 L). The organic layer was combined, washed with city water (145 L), and concentrated under reduced pressure up to about 190 L. Tetrahydrofuran (508 L) was added to the residue to give a tetrahydrofuran solution of DMA (Net 195 kg, yield 97.6%).

### REFERENCE EXAMPLE 2

### N-pivaloyl-p-chloroaniline

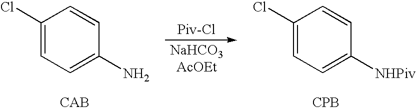

CAB (113 kg, 886 mol), city water (565 L), and sodium bicarbonate (89.3 kg, 1.2 eq) were added to ethyl acetate (1695 L), and pivaloyl chloride (112 kg, 1.05 eq) was added dropwise thereto at 15° C. or lower, and the solution was stirred at about 25° C. for 2 hours. After separating the layers, the organic layer washed with city water (848 L×2), and concentrated under reduced pressure up to about 600 L. Ethylcyclohexane (848 L) was added thereto, and concentrated again under reduced pressure up to about 600 L. The residue was cooled to about 5° C., and stirred to mature for 1 hour. The precipitated crystals were collected by filtration, and dried under reduced pressure to give the title compound (187 kg, yield 99.7%).

### REFERENCE EXAMPLE 3

### 5-chloro-2-pivaloylamino-2′,3′-dimethoxybenzophenone

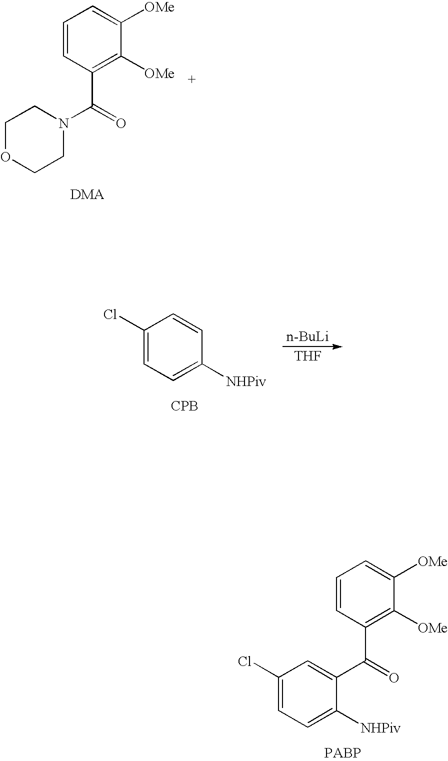

Tetrahydrofuran (516 L) and CPB (164 kg, 775 mol)/tetrahydrofuran (1311 L) solution were added dropwise to 15% n-butyl lithium/n-hexane solution (Net 124 kg) at about −30° C., and stirred for 30 minutes at the same temperature, and then stirred for 2 hours at about 23° C. To the solution was added dropwise DMA/tetrahydrofuran solution (Net 195 kg, 776 mol) at about 23° C., and after stirring for 6 hours at the same temperature, the solution was cooled to about 3° C., and 15% ammonium chloride aqueous solution (697 L) was added thereto and stirred at about 23° C. After separating the layers, the organic layer washed with 15% ammonium chloride aqueous solution (697 L), and then, concentrated under reduced pressure up to about 690 L. The residue was warmed and methanol (1311 L) was added at about 43° C., and then, the mixture was heated up to about 63° C. to confirm the dissolution. After confirming the deposition by adding seed crystals at about 50° C., the solution was cooled and stirred to mature for 1 hour at about 5° C. The precipitated crystals were collected by filtration, and the wet crystals (Net 236 Kg, yield 81.1%) were added to methanol (1888 L). After confirming the dissolution at about 63° C., city water (472 L) was added to the solution under the same temperature. After confirming the deposition by adding seed crystals at about 55° C., the solution was cooled and stirred to mature for 1 hour at about 5° C. The precipitated crystals were collected by filtration, and dried under reduced pressure to give the title compound (235 kg, yield 80.6% (DMA standard)).

### REFERENCE EXAMPLE 4

### 2-amino-5-chloro-2′,3′-dimethoxybenzophenone

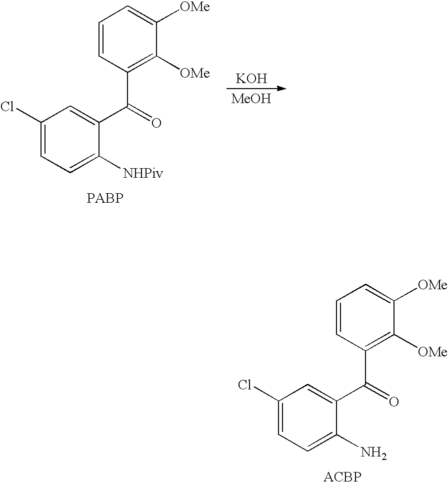

PABP (227 kg, 604 mol) was added to methanol (1363 L) and cooled to about 10° C. After adding potassium hydroxide (141 kg) and city water (148 L) to the solution, the mixture was heated and stirred at about 63° C. for 8 hours. The reaction solution was cooled, and condensed hydrochloric acid (186 kg) and methanol (454 L) were added thereto at 30° C. or lower. The solution was heated, and the deposited solid (KCl) was filtered off at about 63° C. and washed with hot methanol (227 L). The filtrate and washings were combined, and 23 kg of activated charcoal was added thereto with methanol (227 L) at about 63° C. The mixture was stirred for 30 minutes at the same temperature, and filtered, and washed with hot methanol (227 L). The filtrate and washings were combined, and after confirming the crystallization by adding city water (795 L) and seed crystals at about 53° C., the solution was cooled and stirred to mature for 1 hour at about 5° C. The precipitated crystals were collected by filtration, and dried under reduced pressure to give the title compound (168 kg, yield 95.3%).

### REFERENCE EXAMPLE 5

### (S)-2-amino-5-chloro-α-(2,3-dimethoxyphenyl)benzylalcohol

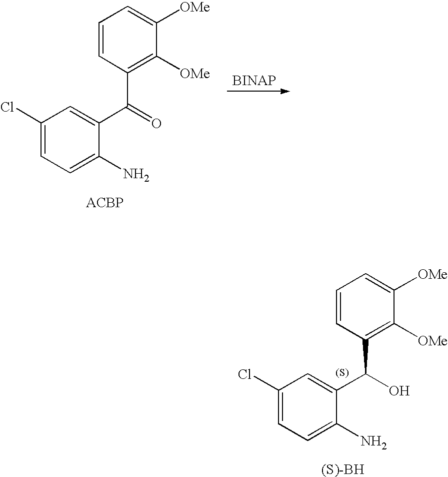

ACBP (198 kg, 679 mol) and tetrahydrofuran (278 kg) were added to isopropyl alcohol (336 kg), and substituted with nitrogen. Ru catalyst Ru₂Cl₄[(S)-DM-BINAP]₂NEt₃ (747 g), (S,S)-diphenylethylenediamine (331 g), tetrahydrofuran (30 kg), potassium hydroxide (1545 g) and isopropyl alcohol (14 kg) were added thereto sequentially, and hydrogen was charged (about 2.6 MPa) at about 60° C., and then stirred for 6 hours. The reaction solution was cooled to about 40° C., activated charcoal (9.9 kg) was added thereto, and stirred for 3 hours. Then, celite (2 kg) was added, and stirred for 10 minutes. The carbon and celite were filtered off, and the filtrate was concentrated under reduced pressure up to about ⅓ in quantity. To the residue was added city water (1190 L), and stirred to mature for 1 hour at about 25° C. The precipitated crystals were collected by filtration, and ethyl acetate (327 kg) was added to dissolve at about 65° C. Then, n-heptane (250 kg) was added, and stirred to mature for 1 hour at about 5° C. The precipitated crystals were collected by filtration, and dried under reduced pressure to give the title compound (128 kg, yield 64.1%).

### REFERENCE EXAMPLE 6

### Ethyl (3R,5S)-7-chloro-1,2,3,5-tetrahydro-1-(3-hydroxy-2,2-dimethylpropyl)-5-(2,3-dimethoxyphenyl)-2-oxo-4,1-benzoxazepin-3-acetate

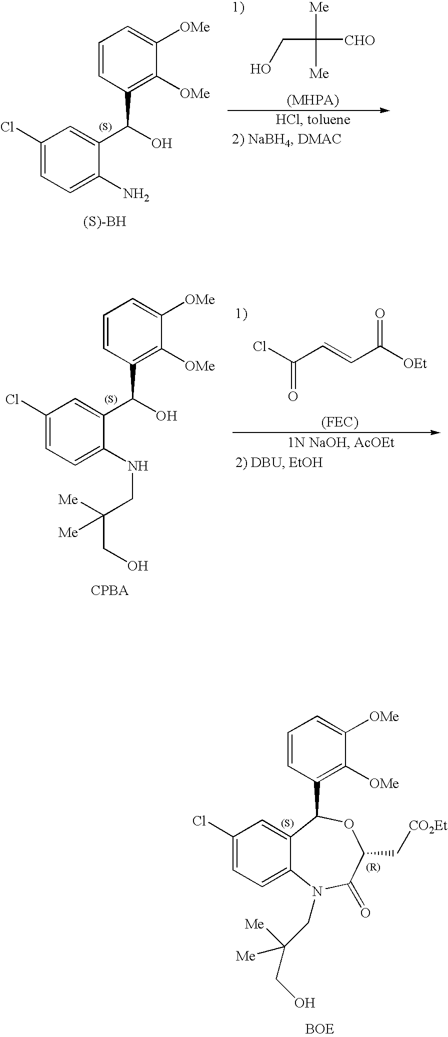

(S)—BH (79.6 kg, 271 mol) was added to toluene (277 kg), and MHPA (33.3 kg, 1.2 eq) and 15 wt % HCl/IPE solution (13.6 kg, 0.2 eq) were added thereto at about 25° C. and then, stirred for 30 minutes. Anhydrous magnesium sulfate (9.8 kg, 0.3 eq) was added, and stirred for 1.5 hours at about 25° C. Then, the mixture was filtered, and washed with toluene (139 kg). The filtrate and washings were combined and cooled, and N,N-dimethylacetamide (29.9 kg), 15 wt % HCl/IPE solution (81.6 kg, 1.2 eq) and sodium borohydride (11.3 Kg)/N,N-dimethylacetamide (127 Kg) solution were added at about 5° C., and stirred for 1 hour at about the same temperature. To the reaction mixture were added 7.7 wt % sodium hydroxide aqueous solution (282 kg) and methanol (63 kg) at 10° C. or lower, and stirred for 1 hour at about 25° C. After separating the layers, the organic layer was washed with city water (239 kg×2) to give a toluene solution of CPBA. This solution was concentrated under reduced pressure up to about 210 L, and ethyl acetate (358 kg) was added thereto, and then concentrated again under reduced pressure up to about 210 L. After adding ethyl acetate (716 kg) and 3.85 wt % sodium hydroxide aqueous solution (424 kg, 1.5 eq), FEC (61.7 kg, 1.4 eq)/ethyl acetate (143 kg) solution was added at about 30° C., and washed in with ethyl acetate (29 kg). After stirring for 1 hour at about 30° C., the layers were separated, and the organic layer washed with 5 wt % sodium bicarbonate aqueous solution (331 kg×2). The organic layer was concentrated under reduced pressure up to about 406 L, ethanol (314 kg) was added thereto, and concentrated again under reduced pressure up to about 400 L. To the residue was added ethanol (126 kg), and DBU (20.8 kg, 0.5 eq) was added at about 60° C., and stirred for 4 hours. After cooling to about 25° C. and stirring for 1 hour, the precipitated crystals were collected by filtration, and dried under reduced pressure to give the title compound (111 kg, yield 80.9%).

### REFERENCE EXAMPLE 7

### (3R,5S)-7-chloro-1,2,3,5-tetrahydro-1-(3-hydroxy-2,2-dimethylpropyl)-5-(2,3-dimethoxyphenyl)-2-oxo-4,1-benzoxazepin-3-acetic Acid

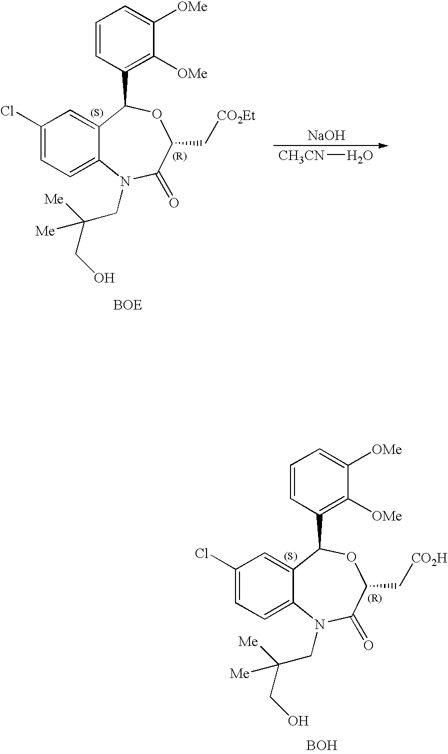

BOE (92 kg, 182 mol) and 1.6 wt % NaOH aqueous solution (560 kg, 1.2 eq) were added to acetonitrile (352 kg), and the solution was stirred for 2 hours under reflux with heating (about 74° C.). After adding 21 wt % hydrochloric acid (44.2 kg, 1.4 eq) at about 50° C. and stirring for 1 hour at the same temperature, the reaction mixture was cooled to about 25° C. and stirred to mature for 1 hour. The precipitated crystals were collected by filtration, and dried under reduced pressure to give the title compound (75.9 kg, yield 87.3%).

The extract of the product of this reaction can be used for next step as it is by adding AcOEt to the reaction solution and extracting after reaction has completed.

### REFERENCE EXAMPLE 8

### (3R,5S)-7-chloro-5-(2,3-dimethoxyphenyl)-1,2,3,5-tetrahydro-1-(3-acetoxy-2,2-dimethylpropyl)-2-oxo-4,1-benzoxazepin-3-acetic Acid

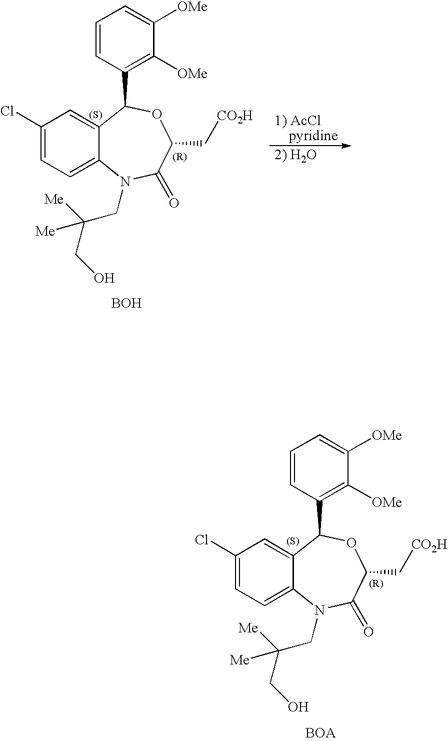

BOH (98.8 kg, 207 mol) and pyridine (89.8 kg, 4.0 eq) were added to ethyl acetate (1144 kg), and acetyl chloride (81.6 kg, 3.5 eq) was added thereto at 5° C. or lower. After reacting at 28° C. to 35° C. for 2 hours, city water (197 kg) was added, and stirred at 40° C. to 44° C. for 2 hours. The layers were separated, and the organic layer washed with 3.5% hydrochloric acid (199 kg) and city water (198 kg×2), and then activated charcoal (2.5 kg) was added and stirred for 30 minutes at 20° C. to 30° C. The activated charcoal was filtered off and washed with ethyl acetate (89 kg), and then concentrated under reduced pressure up to 490 L of the residual volume. To the residue was added n-heptan (534 kg) at 23° C. to 27° C., and stirred to mature at 1° C. to 5° C. with cooling for 2 hours. The precipitated crystals were collected by filtration, and dried under reduced pressure to give the title compound (96.7 kg, yield 90.0%).

### EXAMPLE 1

### 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic Acid

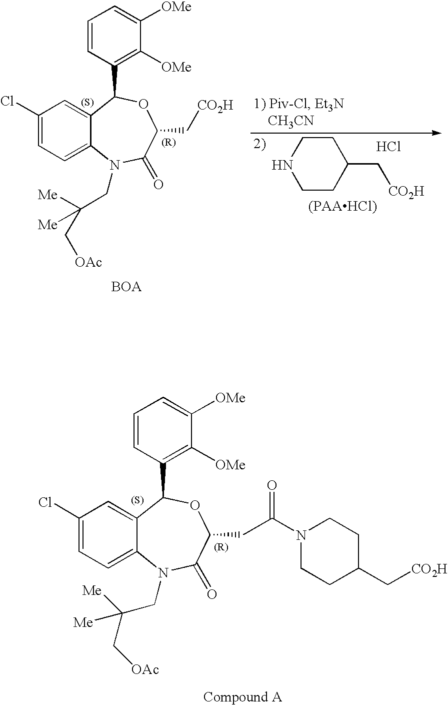

BOA (23.0 kg, 44.2 mol) and triethylamine (4.6 kg, 1.0 eq) were added to acetonitrile (138 L), and pivaloyl chloride (5.8 kg, 1.1 eq) was added thereto at about 0° C. After reacting at 0° C. to 5° C. for 1 hour, PAA.HCL (9.7 kg, 1.2 eq) and triethylamine (6.7 kg, 1.5 eq) were added at the same temperature. After stirring at 20° C. to 28° C. for 30 minutes, 0.5N HCl (46 L) and ethyl acetate (184 L) were added and the layers were separated. The organic layer was washed with 3% brine (46 L×2), and concentrated under reduced pressure to total volume of 140 L. n-Heptane (92 L) was added thereto at 75° C. to 55° C. After cooling to about 5° C. and stirring to mature for 1 hour, the precipitated crystals were collected by filtration, and dried under reduced pressure to give the title compound (26.0 kg, yield 88.4%).

The product of this reaction can be crystallized by adding 0.5N HCl and city water after the reaction has completed.

Purification Process

26.0 kg of the above crystals were dissolved at about 60° C. in a mixture solution of ethanol (164 L) and purified water (19 L), 146 L of purified water was added thereto, and stirred to mature for 1 hour at about 5° C. with cooling. The precipitated crystals were collected by filtration, and dried under reduced pressure to give the title compound (25.0 kg, yield 99.4%, containing dipiperidyl compound: 0.16%, dimer: 0.06%, total related substance (total impurity): 0.4%).

### PREPARATION EXAMPLE 1

[Production of Coating Agent]

224.4 g of Hydroxypropylmethyl cellulose 2910 (TC-5) and 45.0 g of macrogol 6000 were dissolved in 2700 g of purified water. 30.0 g of Titanium oxide and 0.6 g of iron sesquioxide were dispersed in the obtained solution to prepare coating agent.

[Production of Uncoated Tablet]

After 387.5 g of compound A, 2929.5 g of lactose and 930.0 g of corn starch were mixed homogeneously in fluidized bed granulation dryer (FD-5S, Powrex Corporation), aqueous solution in which 139.5 g of hydroxypropylcellulose (HPC-L) was dissolved was sprayed to granulate in the machine, and then dried in the fluidized bed granulation dryer.

The obtained granulated substance was milled by 1.5 mmΦ punching screen using Power Mill grinder (P-3, Showa Chemical Machinery Co., Ltd.) to give sized powder.

192 g of carmellose calcium and 25.6 g of magnesium stearate were added to 3622 g of the obtained sized powder, and were mixed in a tumbler mixer (TM-15S, Showa Chemical Machinery Co., Ltd.) to prepare granules for formulation of tablets. The obtained granules were tabletted (tabletting pressure 7 KN/punch) into tablets at the weight of 300 mg using a 9.5 mmΦ punch with a rotary tablet forming machine (Correct 19K, Kikusui Seisakusho Ltd.) to prepare uncoated tablets.

[Production of Film Coated Tablet]

The above-mentioned coating agent was sprayed to the obtained uncoated tablets in dria coater coating machine (DRC-500, Powrex Corporation) to give 10,000 film coated tablets containing 25 mg of compound A per tablet, whose formulation is as follows.

Formulation of Tablets (Composition Per Tablet):

| Composition | Content (mg) |
|---|---|
| (1) compound A | 25.0 |
| (2) lactose | 189.0 |
| (3) corn starch | 60.0 |
| (4) carmellose calcium | 15.0 |
| (5) hydroxypropylcellulose | 9.0 |
| (6) magnesium stearate | 2.0 |
| total (uncoated tablet) | 300.0 |

Formulation of Film Tablet (Composition Per Tablet):

| | |
|---|---|
| (1) uncoated tablet (film component) | 300.0 |
| (2) hydroxypropylmethyl cellulose 2910 | 7.48 |
| (3) macrogol 6000 | 1.5 |
| (4) titanium oxide | 1.0 |
| (5) iron sesquioxide | 0.02 |
| total | 310.0 |

### PREPARATION EXAMPLE 2

[Production of Coating Agent]

224.4 g of Hydroxypropylmethyl cellulose 2910 (TC-5) and 45.0 g of macrogol 6000 were dissolved in 2700 g of purified water. 30.0 g of Titanium oxide and 0.6 g of iron sesquioxide were dispersed in the obtained solution to prepare coating agent.

[Production of Uncoated Tablet]

After 1550.0 g of compound A, 1767 g of lactose and 930.0 g of corn starch were mixed homogeneously in fluidized bed granulation dryer (FD-5S, Powrex Corporation), aqueous solution in which 139.5 g of hydroxypropylcellulose (HPC-L) was dissolved was sprayed to granulate in the machine, and then dried in the fluidized bed granulation dryer.

The obtained granulated substance was milled by 1.5 mmΦ punching screen using Power Mill grinder (P-3, Showa Chemical Machinery Manufacturing Co., Ltd.) to give sized powder.

192 g of carmellose calcium and 25.6 g of magnesium stearate were added to 3622 g of the obtained sized powder, and were mixed in a tumbler mixer (TM-15S, Showa Chemical Machinery Co., Ltd.) to prepare granules for formulation of tablet. The obtained granules were tabletted (tabletting pressure 7 KN/punch) into tablets at the weight of 300 mg using a 9.5 mmΦ punch with a rotary tablet forming machine (Correct 19K, Kikusui Seisakusho Ltd.) to prepare uncoated tablets.

[Production of Film Coated Tablet]

The above-mentioned coating agent was sprayed to the obtained uncoated tablets in doria coater coating machine (DRC-500, Powrex Corporation) to give 10,000 film coated tablets containing 100 mg of compound A per tablet, whose formulation is as follows.

Formulation of Tablets (Composition Per Tablet):

| Composition | Content (mg) |
|---|---|
| (1) compound A | 100.0 |
| (2) lactose | 114.0 |
| (3) corn starch | 60.0 |
| (4) carmellose calcium | 15.0 |
| (5) hydroxypropylcellulose | 9.0 |
| (6) magnesium stearate | 2.0 |
| total (uncoated tablet) | 300.0 |

Formulation of Film Tablet (Composition Per Tablet):

| | |
|---|---|
| (1) uncoated tablet (film component) | 300.0 |
| (2) hydroxypropylmethyl cellulose 2910 | 7.48 |
| (3) macrogol 6000 | 1.5 |
| (4) titanium oxide | 1.0 |
| (5) iron sesquioxide | 0.02 |
| total | 10.0 |

### PREPARATION EXAMPLE 3

[Production of Coating Agent]

224.4 g of Hydroxypropylmethyl cellulose 2910 (TC-5) and 45.0 g of macrogol 6000 were dissolved in 2700 g of purified water. 30.0 g of Titanium oxide and 0.6 g of iron sesquioxide were dispersed in the obtained solution to prepare coating agent.

[Production of Uncoated Tablet]

After 775.0 g of compound A, 2542 g of lactose and 930.0 g of corn starch were mixed homogeneously in fluidized bed granulation dryer (FD-5S, Powrex Corporation), aqueous solution in which 139.5 g of hydroxypropylcellulose (HPC-L) was dissolved was sprayed to granulate in the machine, and then dried in the fluidized bed granulation dryer.

The obtained granulated substance was milled by 1.5 mmΦ punching screen using Power Mill grinder (P-3, Showa Chemical Machinery Co., Ltd.) to give sized powder.

192 g of carmellose calcium and 25.6 g of magnesium stearate were added to 3622 g of the obtained sized powder, and were mixed in a tumbler mixer (TM-15S, Showa Chemical Machinery Co., Ltd.) to prepare granules for formulation of tablet. The obtained granules were tabletted (tabletting pressure 10 KN/punch) into tablets at the weight of 300 mg using a 9.5 mmΦ punch with a rotary tablet forming machine (Correct 19K, Kikusui Seisakusho Ltd.) to prepare uncoated tablet.

[Production of Film Coated Tablet]

The above-mentioned coating agent was sprayed to the obtained uncoated tablets in doria coater coating machine (DRC-500, Powrex Corporation) to give 10,000 film coated tablets containing 50 mg of compound A per tablet, whose formulation is as follows.

Formulation of Tablets (Composition Per Tablet):

| Composition | Content (mg) |
|---|---|
| (1) compound A | 50.0 |
| (2) lactose | 164.0 |
| (3) corn starch | 60.0 |
| (4) carmellose calcium | 15.0 |
| (5) hydroxypropylcellulose | 9.0 |
| (6) magnesium stearate | 2.0 |
| total (uncoated tablet) | 300.0 |

Formulation of Film Tablet (Composition Per Tablet):

| | |
|---|---|
| (1) uncoated tablet (film component) | 300.0 |
| (2) hydroxypropylmethyl cellulose 2910 | 7.48 |
| (3) macrogol 6000 | 1.5 |
| (4) titanium oxide | 1.0 |
| (5) iron sesquioxide | 0.02 |
| total | 310.0 |

### PREPARATION EXAMPLE 4

[Production of Coating Agent]

224.g of Hydroxypropylmethyl cellulose 2910 (TC-5) and 45.0 g of macrogol 6000 were dissolved in 2700 g of purified water. 30.0 g of Titanium oxide and 0.6 g of iron sesquioxide were dispersed in the obtained solution to prepare coating agent.

[Production of Uncoated Tablet]

After 1550.0 g of compound A, 1767 g of lactose and 930.0 g of corn starch were mixed homogeneously in fluidized bed granulation dryer (FD-5S, Powrex Corporation), aqueous solution in which 139.5 g of hydroxypropylcellulose (HPC-L) was dissolved was sprayed to granulate in the machine, and then dried in the fluidized bed granulation dryer.

The obtained granulated substance was milled by 1.5 mmΦ punching screen using Power Mill grinder (P-3, Showa Chemical Machinery Manufacturing Co., Ltd.) to give sized powder.

192 g of carmellose calcium and 25.6 g of magnesium stearate were added to 3622 g of the obtained sized powder, and were mixed in a tumbler mixer (TM-15S, Showa Chemical Machinery Co., Ltd.) to prepare granules for formulation of tablet. The obtained granules were tabletted (tabletting pressure 7 KN/punch) into tablets at the weight of 150 mg using a 7.5 mmΦ punch with a rotary tablet forming machine (Correct 19K, Kikusui Seisakusho Ltd.) to prepare uncoated tablets.

[Production of Film Coated Tablet]

The above-mentioned coating agent was sprayed to the obtained uncoated tablets in doria coater coating machine (DRC-500, Powrex Corporation) to give 20,000 film coated tablets containing 50 mg of compound A per tablet, whose formulation is as follows.

Formulation of Tablets (Composition Per Tablet):

| Composition | Content (mg) |
|---|---|
| (1) compound A | 50.0 |
| (2) lactose | 57.0 |
| (3) corn starch | 30.0 |
| (4) carmellose calcium | 7.5 |
| (5) hydroxypropylcellulose | 4.5 |
| (6) magnesium stearate | 1.0 |
| total (uncoated tablet) | 150.0 |

Formulation of Film Tablet (Composition Per Tablet):

| | |
|---|---|
| (1) uncoated tablet (film component) | 150.0 |
| (2) hydroxypropylmethyl cellulose 2910 | 3.74 |
| (3) macrogol 6000 | 0.75 |
| (4) titanium oxide | 0.5 |
| (5) iron sesquioxide | 0.01 |
| total | 155.0 |

### PREPARATION EXAMPLE 5

[Production of Coating Agent]

2244 g of Hydroxypropylmethyl cellulose 2910 (TC-5) and 450.0 g of macrogol 6000 were dissolved in 27000 g of purified water. 300.0 g of Titanium oxide and 6.0 g of iron sesquioxide were dispersed in the obtained solution to prepare coating agent.

[Production of Uncoated Tablet]

After 4330 g of compound A, 4872 g of lactose and 2580 g of corn starch were mixed homogeneously in fluidized bed granulation dryer (FD-5S, Powrex Corporation), aqueous solution in which 387.0 g of hydroxypropylcellulose (HPC-L) was dissolved was sprayed to granulate in the machine, and then dried in the fluidized bed granulation dryer.

The obtained granulated substance was milled by 1.5 mmΦ punching screen using Power Mill grinder (P-3, Showa Chemical Machinery Manufacturing Co., Ltd.) to give sized powder.

1688 g of carmellose calcium and 225.0 g of magnesium stearate were added to 31840 g of the obtained sized powder, and were mixed in a tumbler mixer (200 L, Suchiro Chemical Machinery Co., Ltd.) to prepare granules for formulation of tablets. The obtained granules were tabletted (tabletting pressure 15 KN/punch) into tablets at the weight of 300 mg using a 9.5 mmΦ punch with a rotary tablet forming machine (Aquarius 36K, Kikusui Seisakusho Ltd.) to prepare uncoated tablets.

[Production of Film Coated Tablet]

The above-mentioned coating agent was sprayed to the obtained uncoated tablets in film coating machine (HCFS-100N, Freund) to give 100,000 film coated tablets containing 100 mg of compound A per tablet, whose formulation is as follows.

Formulation of Tablets (Composition Per Tablet):

| Composition | Content (mg) |
|---|---|
| (1) compound A | 100.0 |
| (2) lactose | 114.0 |
| (3) corn starch | 60.0 |
| (4) carmellose calcium | 15.0 |
| (5) hydroxypropylcellulose | 9.0 |
| (6) magnesium stearate | 2.0 |
| total (uncoated tablet) | 300.0 |

Formulation of Film Tablet (Composition Per Tablet):

| | |
|---|---|
| (1) uncoated tablet (film component) | 300.0 |
| (2) hydroxypropylmethyl cellulose 2910 | 7.48 |
| (3) macrogol 6000 | 1.5 |
| (4) titanium oxide | 1.0 |
| (5) iron sesquioxide | 0.02 |
| total | 310.0 |

### INDUSTRIAL APPLICABILITY

The present invention provides an industrial process for producing, with high yield, an aliphatic cyclic carboxamide having carboxyl group of high quality which is useful as medicine during the shorter steps by reacting carboxylic acid anhydride with aliphatic cyclic secondary amine having carboxyl group, so the present invention is useful, for example, in the pharmaceutical industry.

1. A process for producing an aliphatic cyclic carboxamide having carboxyl group, which comprises reacting tertiary carboxylic acid anhydride and aliphatic cyclic secondary amine having carboxyl group.

2. A process for producing an aliphatic cyclic carboxamide having carboxyl group, which comprises reacting carboxylic acid anhydride obtained by reacting carboxylic acid and tertiary carboxylic acid halide with aliphatic cyclic secondary amine having carboxyl group.

3. The process according to claim 2, wherein the tertiary carboxylic acid halide is pivaloyl chloride.

4. The process according to claim 2, wherein the carboxylic acid is a compound represented by the formula: 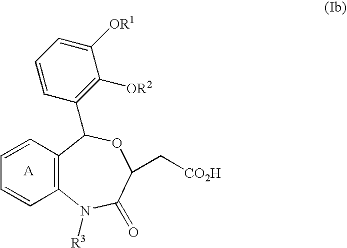
wherein R¹ and R² each independently denote a lower alkyl group, R³ denotes a lower alkyl group which may be substituted with hydroxyl group or an alkanoyloxy group, and ring A denotes a benzene ring which may be substituted with a halogen atom, or a salt thereof.

5. The process according to claim 2, wherein the carboxylic acid is (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid or a salt thereof.

6. The process according to claim 1, wherein the aliphatic cyclic secondary amine having carboxyl group is a compound represented by the formula: 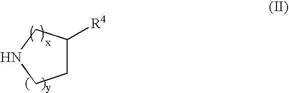
wherein x denotes an integer of 1, 2 or 3; y denotes an integer of 0, 1, or 2; and R⁴ denotes a group represented by the formula —(CH₂)_{z}—CO₂H [wherein z denotes an integer of 0, 1, 2, or 3], or a salt thereof.

7. The process according to claim 1, wherein the aliphatic cyclic secondary amine having carboxyl group is piperidine-4-acetic acid or a salt thereof.

8. A process for producing 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid or a salt thereof, which comprises reacting (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic pivalic anhydride or a salt thereof with piperidine-4-acetic acid or a salt thereof.

9. A process for producing 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid or a salt thereof, which comprises reacting (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic pivalic anhydride or a salt thereof with piperidine-4-acetic acid or a salt thereof, followed by subjecting the resulting compounds to recrystallization.

10. A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to claim 9, wherein the content of dipiperidyl compound is less than 0.5% of total weight of the composition.

11. A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to claim 9, wherein the content of dimer is less than 0.5% of total weight of the composition.

12. A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to claim 9, wherein the content of dimer is less than 0.3% of total weight of the composition.

13. A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to claim 9, wherein any impurities exceeding 0.2% of total weight of the composition other than dipiperidyl compound or dimer are not contained.

14. A composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, which is obtained by the process according to claim 9, wherein the content of total impurity is less than 1.0% of total weight of the composition.

15. A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of dipiperidyl compound is less than 0.5% of total weight of the composition, to a human in need thereof.

16. A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of dimer is less than 0.5% of total weight of the composition, to a human in need thereof.

17. A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of dimer is less than 0.3% of total weight of the composition, to a human in need thereof.

18. A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein any impurities exceeding 0.2% of total weight of the composition other than dipiperidyl compound or dimer are not contained, to a human in need thereof.

19. A method for preventing and/or treating hyperlipidemia, familial hypercholesterolemia, organ failure or organ dysfunction and a method for protecting skeletal muscle, which comprises administering a composition of 1-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid, wherein the content of total impurity is less than 1.0% of total weight of the composition, to a human in need thereof.
